(19) United States
(12) Patent Application Publication
(10) Pub. No.: US 2014/0227141 A1
(43) Pub. Date: Aug. 14, 2014

(54) Vaporizing Device

(71) Applicant: Pro-Iroda Industries, Inc., Taichung City (TW)

(72) Inventor: Wei-Long Chen, Taichung City (TW)

(73) Assignee: Pro-Iroda Industries, Inc., Taichung City (TW)

(21) Appl. No.: 14/071,815

(22) Filed: Nov. 5, 2013

(30) Foreign Application Priority Data
Feb. 8, 2013 (TW) 102105179

(51) Int. Cl. A61L 9/03

(52) U.S. Cl. CPC A61L 9/037
USPC 422/125

(57) ABSTRACT

A vaporizing device includes a reservoir defining a receiving space. A wick is engaged in the receiving space. A heat conducting member caps on the reservoir and includes a hole extending therethrough connecting and in communication with the receiving space and including a recess. The recess is delimited by an inner ridge, an outer ridge and a floor side of the heat conducting member. The outer ridge is radially spaced from the inner edge. The floor side extends radially between the inner and outer edges. The inner ridge delimits an opening corresponding to the hole. The wick has an end extending through the hole and the opening. Liquid to be vaporized by the vaporizing device is filled in the recess and fuel is filled in receiving space.

### BACKGROUND OF THE INVENTION

1. Field of the Invention

The present invention relates to a vaporizing device and, particularly to a vaporizing device that can vaporize liquid in a faster and safer manner.

2. Description of the Related Art

TW Patent No. M422422 shows a fragrance oil lamp. The fragrance oil lamp includes an oil reservoir, a wick, and a heating device disposed outside the oil reservoir and having first and second sections surrounding a distal end and the middle of the wick respectively. According to the disclosure, heat conducts on the first section of the heating device, and the second section of the heating device apply the heat to the middle section of the wick. Therefore, the fragrance oil lamp is claimed to vaporize the oil effectively. The present invention is therefore, intended to provide a vaporizing device that can vaporize oil in a faster and safer manner.

### SUMMARY OF THE INVENTION

According to the present invention, a vaporizing device includes a reservoir defining a receiving space. A wick is engaged in the receiving space. A heat conducting member caps on the reservoir and includes a hole extending therethrough connecting and in communication with the receiving space and includes a recess. The recess is delimited by an inner ridge, an outer ridge and a floor side of the heat conducting member. The outer ridge is radially spaced from the inner edge. The floor side extends radially between the inner and outer edges. The inner ridge delimits an opening corresponding to the hole. The wick has an end extending through the hole and the opening. Liquid to be vaporized by the vaporizing device is filled in the recess and fuel is filled in receiving space.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a vaporizing device that can vaporize liquid in a faster and safer manner.

Other objects, advantages, and new features of the present invention will become apparent from the following detailed description of the invention when considered in conjunction with the accompanied drawings.

### BRIEF DESCRIPTION OF THE DRAWINGS

FIG. 1 is an exploded, perspective view of a vaporizing device in accordance with the present invention.

FIG. 2 is a perspective view of the vaporizing device of the present invention.

FIG. 3 is a cross-sectional view of FIG. 2, showing the vaporizing device in use.

FIG. 4 is a continued cross-sectional view of FIG. 3, showing the vaporizing device in use.

FIG. 5 is a perspective view of a wind shield, with the vaporizing device of the present invention further including the wind shield engagable therewith.

FIG. 6 is a continued perspective view of FIG. 5, showing the wind shield engaged with the vaporizing device.

FIG. 7 is a cross-sectional view of FIG. 6, showing the vaporizing device in use.

FIG. 8 is a continued cross-sectional view of FIG. 7, showing the vaporizing device in use.

FIG. 9 is a perspective view of a flame extinguisher, with the vaporizing device of the present invention further including the flame extinguisher engagable therewith.

FIG. 10 is a continued perspective view of FIG. 9, showing the flame extinguisher engaged with the vaporizing device.

### DETAILED DESCRIPTION OF THE INVENTION

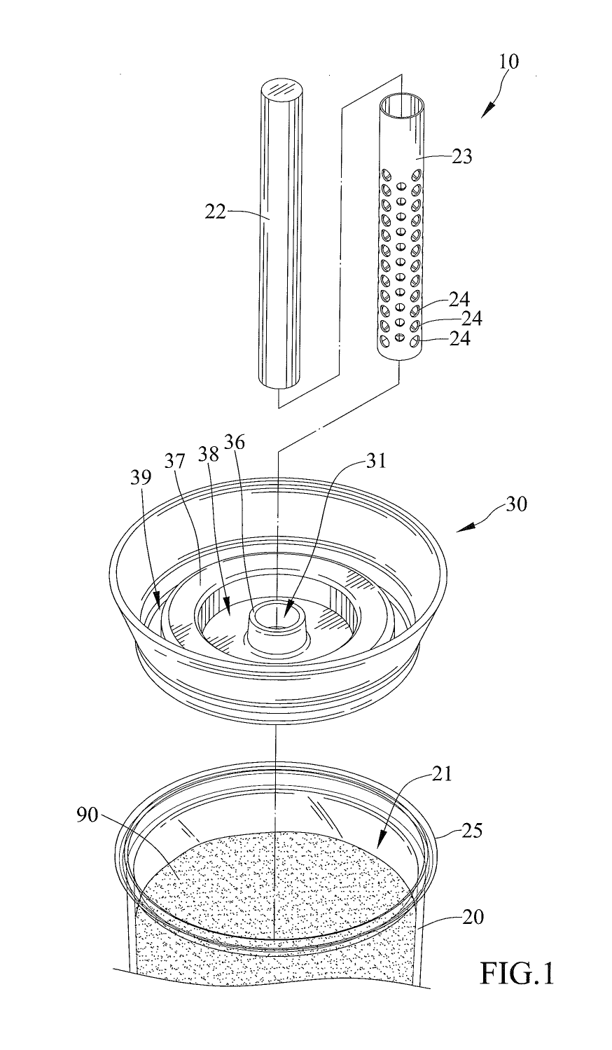

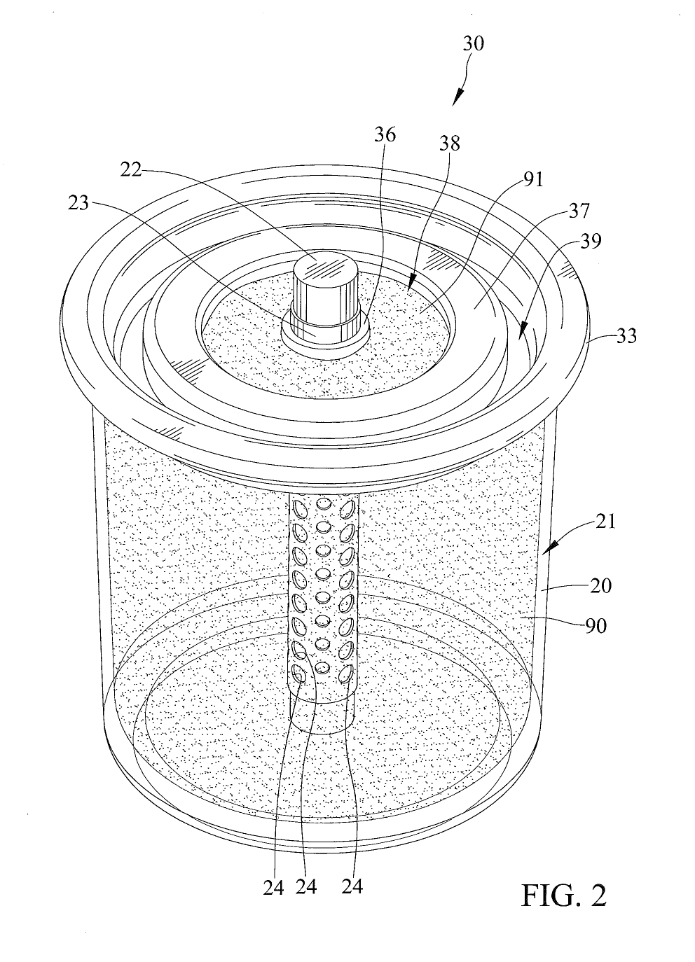

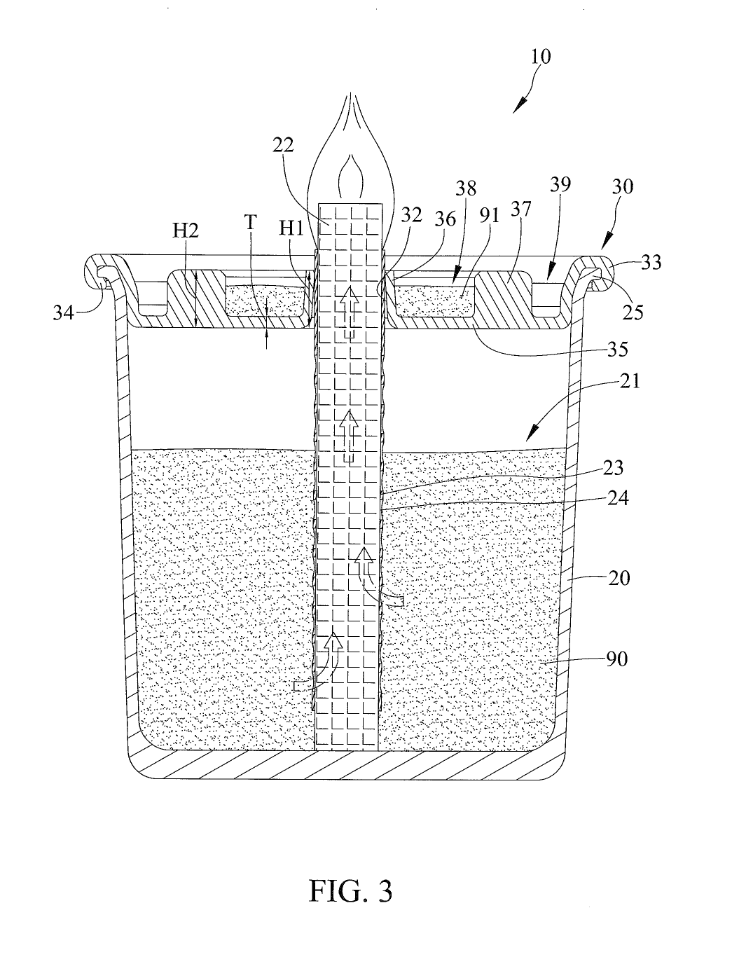

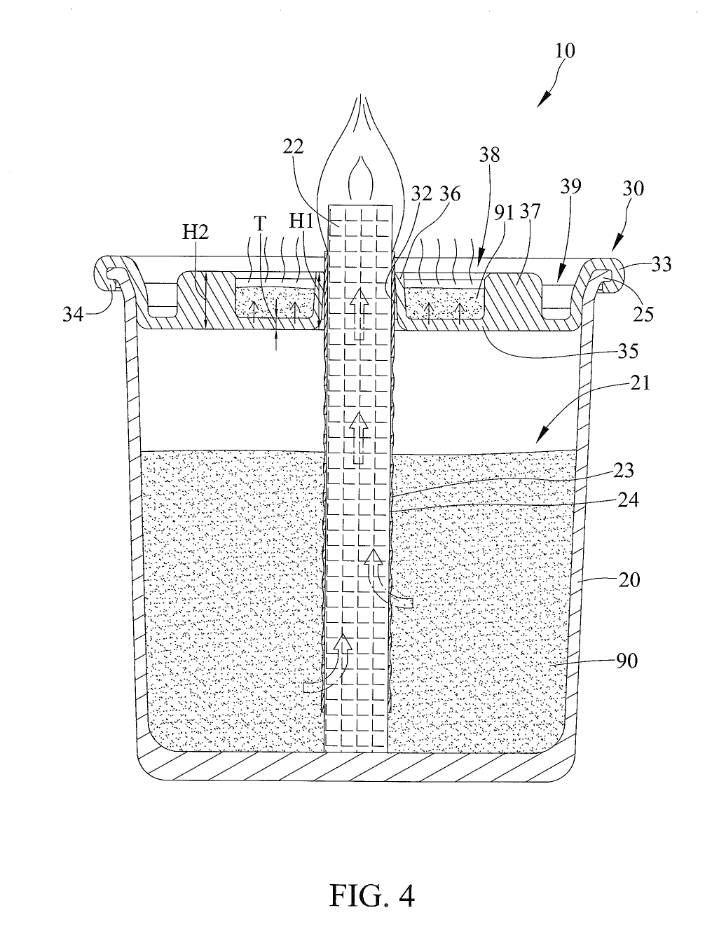

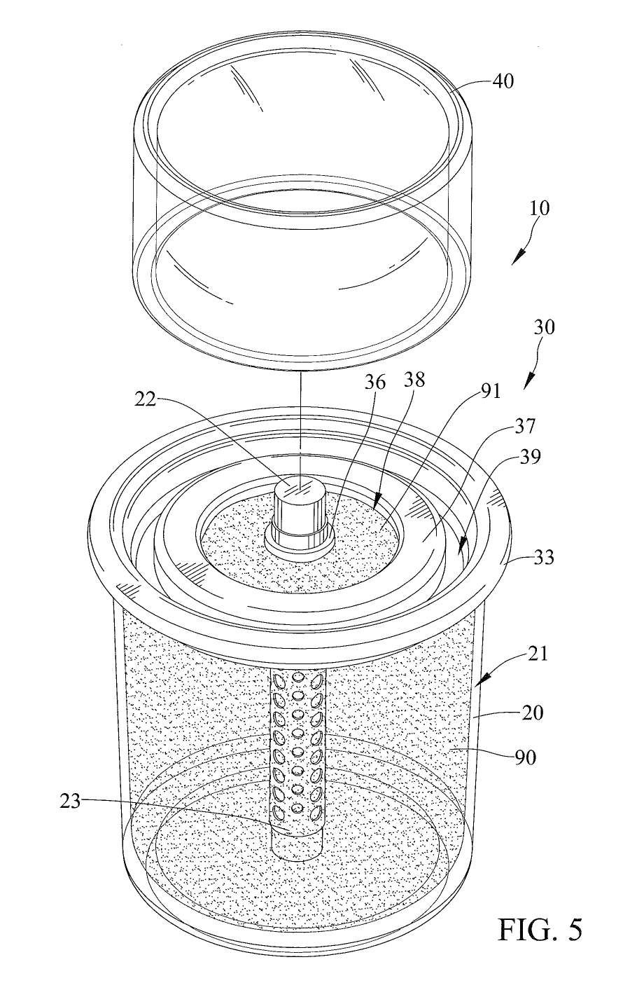

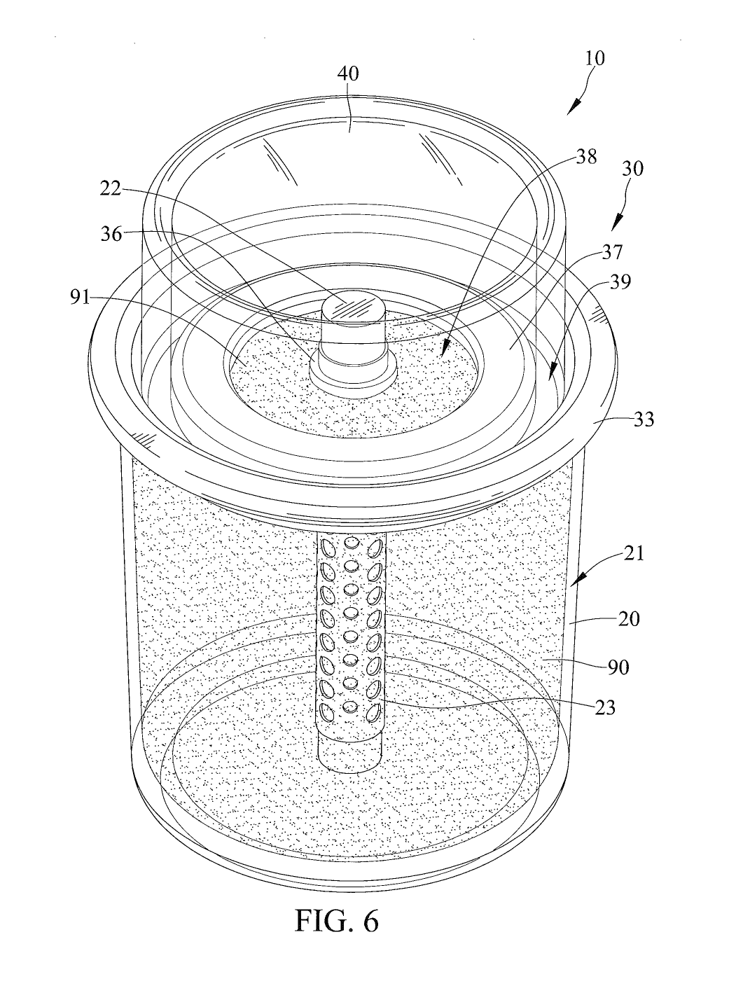

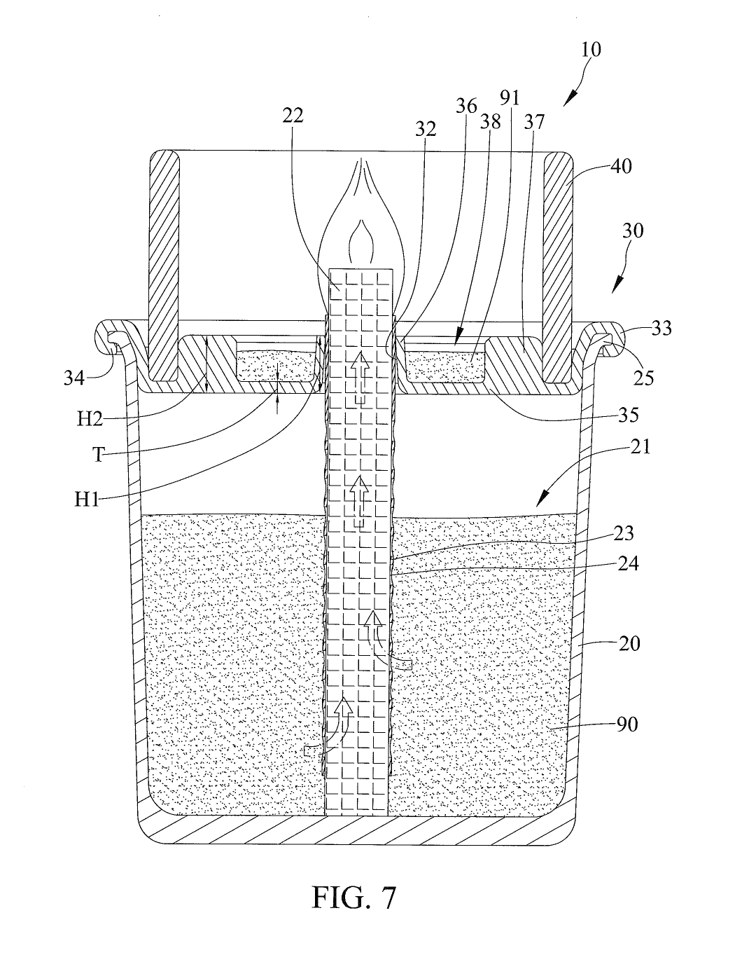

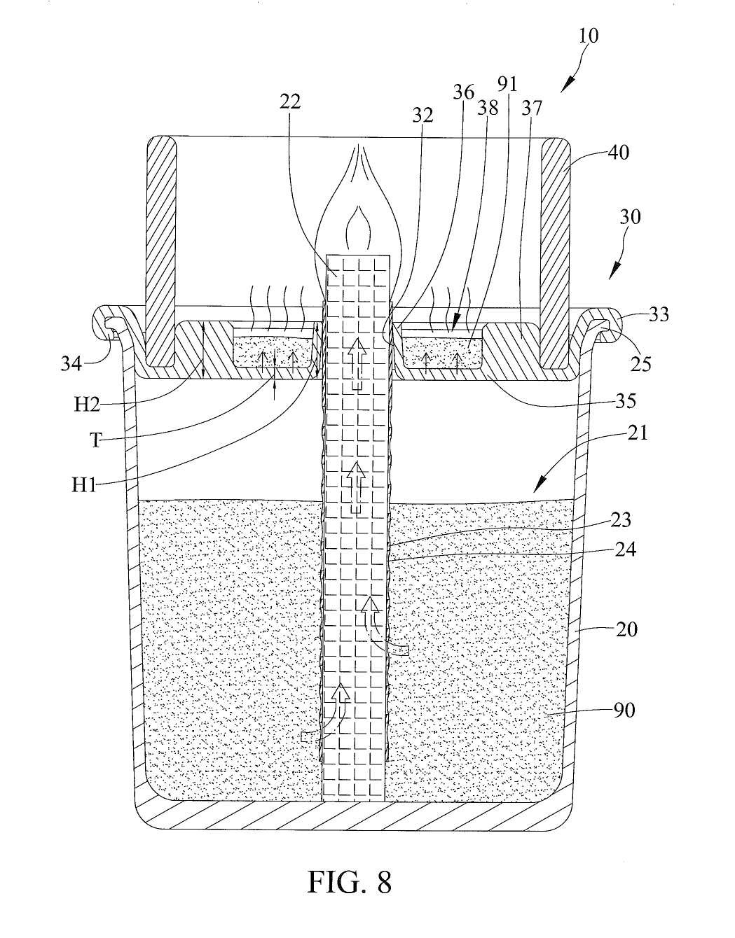

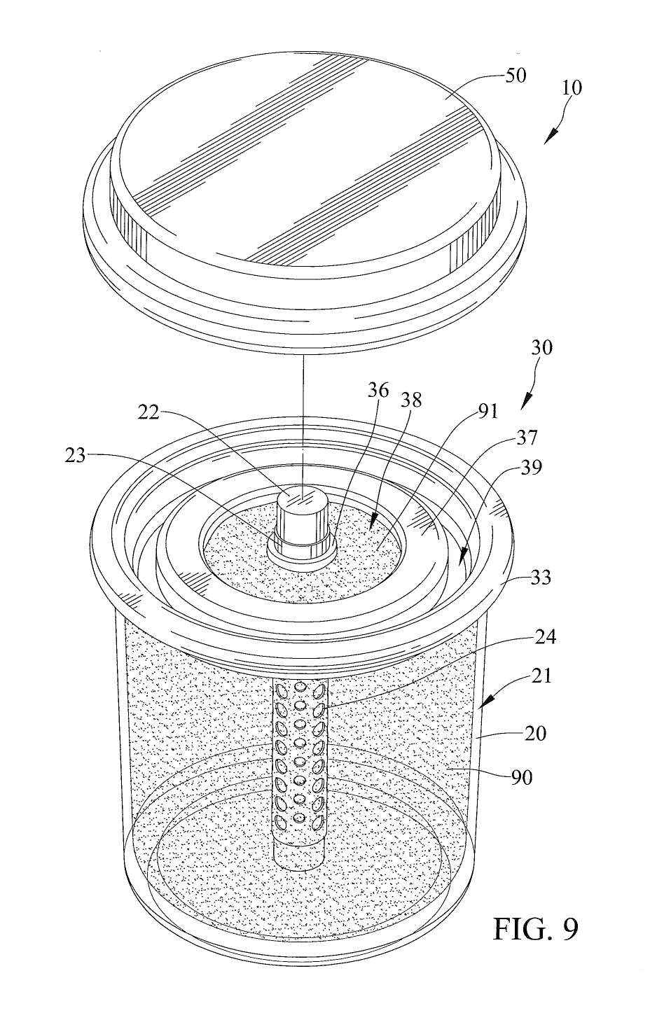

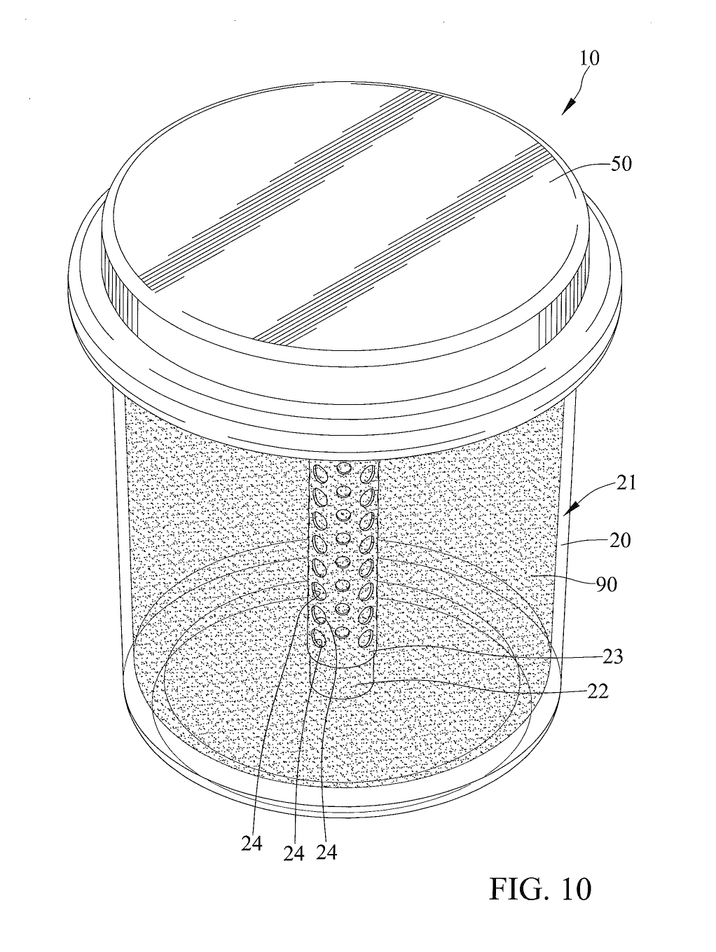

FIGS. 1 through 10 show a vaporizing device 10 in accordance with the present invention.

The vaporizing device 10 includes a reservoir 20 defining a receiving space 21. Fuel is filled in receiving space 21. A wick 22 is engaged in the receiving space 21. The wick 22 is inserted into a sleeve 23, with the wick 22 having a circumferential periphery and the sleeve 23 having an inner periphery abutting against the circumferential periphery of the wick 22. The wick 22 has two distal ends exposed outside the sleeve 23. The sleeve 23 includes a plurality of orifices 24 extending radially therethrough. The wick 22 and the sleeve 23 are both thermal conductive. The wick 22 has a first longitudinal length and the sleeve 23 has a second longitudinal length shorter than the first longitudinal length.

A heat conducting member 30 caps on the reservoir 20. The heat conducting member 30 is securely connected to a rim 25 of the reservoir 20. The rim 25 of the reservoir 20 forms a flange structure, and the heat conducting member 30 has an outer peripheral edge 33 forming a hook structure 34 receiving the flange structure. The heat conducting member 30 is then securely connected to the reservoir 20. The heat conducing member 30 is made of metal. The heat conducting member 30 includes a hole 31. The hole 31 extends through the heat conducting member 30 and connects and in communication with the receiving space 21. The heat conducting member 30 includes a recess 38. Liquid to be vaporized by the vaporizing device 10 is filled in the recess 38. The recess 38 is delimited by an inner ridge 36, an outer ridge 37 and a floor side of the heat conducting member 30. The outer ridge 37 is radially spaced from the inner edge 36. The floor side extends radially between the inner and outer edges 36 and 37. The heat conducting member 30 is of a shape 35 that has a top including the inner and outer ridges 36 and 37 protruding outward therefrom, and a bottom, opposite to the top, including the inner ridge 36 having a first height H1 from the bottom and the outer ridge 37 having a second height H2 from the bottom and the floor side having a thickness T in a height direction of inner and outer ridges 36 and 37 from the bottom, respectively. The thickness T is smaller than the first and second heights H1 and H2. As such, the recess 38 forms a cavity. In addition, the first and second heights H1 and H2 are substantially equal. The inner ridge 36 delimits an opening corresponding to the hole 31. The wick 22 has an end extending through the hole 31 and the opening. The heat conducting member 30 has an inner peripheral edge 32 defining a lateral side of the inner ridge 36, and the sleeve 23 has an outer periphery abutting against the inner peripheral edge of the heat conducting member 30. The heat conducting member 30 includes a slot 39. The slot 39 is delimited between the outer ridge 37 and the outer peripheral edge 33 of the heat conducting member 30. The slot 39 extends radially outside the recess 38.

A wind shield 40 is releasably disposed on the heat conducting member 30, with the wind shield 40 including a first distal end received in the slot 39. The wind shield 40 has a height greater than a length of the wick 22 exposed outside the hole 31 of the heat conducting member 30. The wind shield 40 is transparent that it is allowed to be seen through. The wind shield 40 is hollow and includes a second distal end opposite to the first distal end, with the first and second distal ends forming two open ends. The wind shield 40 protects the flame from being put out by the wind, and since the second distal end forms an opening the flame will not subject to suffer a poor oxygen circulation.

A flame extinguisher 50 releasably caps on the heat conducting member 30 for putting out a flame on the vaporizing device 10.

In use of the vaporizing device, the wick 22 draws the fuel in the receiving space 21 by capillary action up to a flame. The fuel can flow through the plurality of orifices 24. Therefore, the wick 22 can draw the fuel that flows through the plurality of orifices 24.

In view of the forgoing, the liquid to be vaporized is not burnt by the flame. Rather, the flame conducts heat to the heat conducting member 30. The liquid is then vaporized by the heat. The wick 22, the sleeve 23, and the heat conducting member 30 are abutted against each other and they all have good thermal conductivity. Furthermore, since the heat conducting member 30 has a good thermal conductivity, the liquid can vaporize at a faster speed as it gets hotter. In addition, a tight abutment between the outer periphery of the sleeve 23 and the inner peripheral edge of the heat conducting member 30 prevent the flame accidentally burns the fuel.

While the specific embodiments have been illustrated and described, numerous modifications come to mind without significantly departing from the spirit of invention, and the scope of invention is only limited by the scope of accompanying claims.

What is claimed is:

1. A vaporizing device comprising:
a reservoir defining a receiving space;
a wick engaged in the receiving space;
a heat conducting member capping the reservoir and including a hole extending therethrough connecting and in communication with the receiving space and including a recess, with the recess delimited by an inner ridge, an outer ridge and a floor side of the heat conducting member, with the outer ridge radially spacing from the inner edge, with the floor side extending radially between the inner and outer edges, with the inner ridge delimiting an opening corresponding to the hole;
wherein the wick has an end extending through the hole and the opening, and wherein liquid to be vaporized by the vaporizing device is filled in the recess and fuel is filled in receiving space.

2. The vaporizing device as claimed in claim 1, wherein the wick is inserted into a sleeve, with the wick having a circumferential periphery and the sleeve having an inner periphery abutting against the circumferential periphery of the wick.

3. The vaporizing device as claimed in claim 2, wherein the heat conducting member has an inner peripheral edge defining a lateral side of the inner ridge, and the sleeve has an outer periphery abutting against the inner peripheral edge of the heat conducting member.

4. The vaporizing device as claimed in claim 2, wherein the sleeve includes a plurality of orifices extending radially therethrough.

5. The vaporizing device as claimed in claim 2, wherein the wick has a first longitudinal length and the sleeve has a second longitudinal length shorter than the first longitudinal length.

6. The vaporizing device as claimed in claim 5, wherein the wick has two distal ends exposed outside the sleeve.

7. The vaporizing device as claimed in claim 3, wherein the heat conducting member is securely connected to a rim of the reservoir.

8. The vaporizing device as claimed in claim 7, wherein the rim of the reservoir forms a flange structure, and the heat conducting member has an outer peripheral edge forming a hook structure receiving the flange structure.

9. The vaporizing device as claimed in claim 8, wherein the heat conducting member includes a slot extending radially outside the recess, with the slot delimited between the outer ridge and the outer peripheral edge of the heat conducting member.

10. The vaporizing device as claimed in claim 9 further comprising a wind shield releasably disposed on the heat conducting member, with the wind shield including a first distal end received in the slot, and wherein the wind shield has a height greater than a length of the wick exposed outside the hole of the heat conducting member.

11. The vaporizing device as claimed in claim 10, wherein the wind shield is hollow and includes a second distal end opposite to the first distal end, with the first and second distal ends forming two open ends.

12. The vaporizing device as claimed in claim 1 further comprising a flame extinguisher releasably capping the heat conducting member for putting out a flame on the vaporizing device.

13. The vaporizing device as claimed in claim 1, wherein the heat conducing member is made of metal.

14. The vaporizing device as claimed in claim 1, wherein the heat conducting member has a bottom, the inner ridge has a first height from the bottom, the outer ridge has a second height from the bottom, and the floor side has a thickness in a height direction of inner and outer ridges from the bottom, with the thickness smaller than the first and second heights.

15. The vaporizing device as claimed in claim 14, wherein the first and second heights are substantially equal.
